(19) United States
(12) Patent Application Publication
(10) Pub. No.: US 2004/0209795 A1
(43) Pub. Date: Oct. 21, 2004

(54) VOC-free microemulsions

(76) Inventor: Florin Joseph Vlad, Annandale, NJ (US)

Correspondence Address: WINSTON & STRAWN, PATENT DEPARTMENT, 1400 L STREET, N.W., WASHINGTON, DC, 20005-3502, US

(21) Appl. No.: 10/421,216

(22) Filed: Apr. 21, 2003

(51) Int. Cl. A61K 7/46

(52) U.S. Cl. 512/4

(57) ABSTRACT

The present invention provides a VOC-free crystal-clear oil-in-water microemulsion containing up to 30% w/w of an oil, a surfactant svstemn containing a majority of ionic surfactants and a minority of non-ionic surfactants, a w/w oil/surfactant system ratio above 1, up to 10% w/w of a solubilizing-aid ingredient, and at least 60% of water. The present invention concerns also the articles associated with these microemulsions.

### BACKGROUND ART

The present invention relates to the field of perfumer and more precisely it provides a perfume or a perfuming composition in the form of a highly transparent VOC-free microemulsion.

Generally speaking, water-based microemulsions containing a perfume have been already reported in the prior art. Frequently, said microemulsions, in order to facilitate the dispersion of the perfume, which is an oil, contain large amounts of short chain alcohols or other VOC compounds. The problem of said microemulsions is that VOC are nowadays considered as undesired for various reasons.

In general terms, VOC-free microemulsions that may be useful for the perfumery industry have already been described in the prior art. Generally, in formulating such microemulsions is it important to increase the total amount of surfactants, in order to obviate the absence of VOCs, and in particular of ethanol; otherwise the final emulsions display a lack of clarity and/or stability problems, and this is not acceptable. However, the increase of the amount of surfactants in the final microemulsions results in a product containing a surfactant system that is in large excess with respect of the perfume; obviously, this is also a disadvantage for the final product.

Therefore, there is still a need for a perfume, or perfuming product, in the form of a microemulsion that is free of VOCs, and is able to associate a priced crystal-clear appearance, and possibly also a prolonged stability, with a content in surfactants which does not exceed the perfume content.

### SUMMARY OF THE INVENTION

The present invention provides a VOC-free oil-in-water microemulsion which contains, in the one hand, a solubilizing-aid ingredient, and, in the other hand, at least as much oil as surfactants; said microemulsion also possesses a high transparency and possibly a good to excellent stability over a wide temperature range.

Other aspects of the invention are the use of said microemulsion as perfuming ingredient and a consumer article associated with said microemulsion.

### DETAILED DESCRIPTION OF THE PREFERRED EMBODIMENTS

We have now surprisingly discovered that the object of the present invention, cited above, can be achieved by using a suitable amount of an appropriate solubilizing-aid ingredient, in addition to the classical ingredients, namely a perfume, a surfactant system and water.

Therefore, a first object of the present invention is a VOC-free oil-in-water (o/w) microemulsion comprising:

A) from 3 to 30% w/w of an oil having a surface tension, at 25° C., of at least 25 mN/m;

B) a surfactant system, comprising one ore more ionic surfactants, such as anionic, cationic and/or amphoteric surfactants, in a quantity of at least 50% w/w of the surfactant system, and one or more non-ionic surfactant, in a quantity of at most 50% w/w of the surfactant system; the w/w oil/surfactant system ratio being comprised between 1 and 3;

C) from 0.1 to 10% w/w of a solubilizing-aid ingredient selected from the group consisting of the ammonium, alkaline and alkaline earth salts of C₁ to C₁₅ mono- and di-carboxylic acids derivatives, bicarbonates, halogenates, thiocyanates, and mixtures of said salts; and

D) at least 60% of water;

said microemulsion having a clarity comprised between 0 and 90 NTU, when measured between 400 and 600 nm in a 2.5 cm cell at 25° C.

The abbreviation w/w represents the weight to weight ratio, as the ratio between the weight of a specific ingredient and the weight of the microemulsion.

By “VOC” we mean here the Volatile Organic Compounds as defined by the Environmental Protection Agency, and in particular we mean C₁-C₅ alkanols, such as ethanol, or C₁-C₅ alkandiols, such as ethylene glycol.

By “microemulsion” we mean here an emulsion that form spontaneously and has a droplet size comprised between 10 and 150 nm, at a temperature comprised between 0° and 80° C. However, according to a particular embodiment of the invention, the present microemulsion has a droplet size comprised between 10 and 60 nm, or even between 10 and 40 nm, at a temperature comprised between 0° and 80° C.

By “clarity” we mean here the measure of the light scattered, at an angle of 90°, by the invention's microemulsion. According to a praised embodiment of the invention, the microemulsion has a clarity comprised between 0 and 50 NTU when measured in the same conditions as specified above.

In a particular embodiment of the invention, a specific range of surface tension can also characterizes the invention's microemulsion. Indeed, according to a particular embodiment of the invention the microemulsions have a surface tension, measured at 25° C., of at most 30 mN/m, or even below 28 mN/m. Said microemulsions can contain large amounts of low polar oils.

The possibility to have clear VOC-free microemulsion containing large amounts of low polar oils was an unexpected result. Indeed, it is known to a person skilled in the art that it is particularly difficult to dissolve low polar oils in water, especially in large amounts, without the use of some VOCs or oil/surfactant ratio below 1.

As mentioned above, the invention's microemulsions can also display a very good stability, e.g. phase separation is not observed within a reasonable frame of time. Indeed, the invention's microemulsions are commonly stable for at least 30 days, at temperatures comprised between 2 ° and 60° C. Furthermore, in some cases nearly thermodynamic stability, e.g. more than 6 months at temperatures comprised between 2° and 45° C., was achieved.

However, it has to be mentioned that the range of temperatures in which the invention's microemulsion shows very good stability is a function of the amount, as well as the exact nature, of the oil, surfactant system and solubilizing-aid ingredient used. Therefore in some cases it is possible that the stability temperature range of the named microemulsions may be narrower, e.g. from 5° to 45° C. only, or wider, e.g. from 0° to 80° C.

By “oil” we mean here a lipophilic organic liquid which is essentially insoluble in water. An example of a suitable oil is a liquid which comprises at least 75% w/w, or even at least 90% w/w, of a perfume or a perfuming composition. Said oil may also consist of a perfume.

As the perfume or perfuming composition there can be used any perfuming ingredient or, as happens more often, any mixture of perfuming ingredients currently used in perfumery, e.g. of compounds capable of imparting an hedonic olfactive effect to the composition in which they are added. Said perfuming ingredients can be of natural or synthetic origin. Although a detailed description of said perfuming ingredients would be in any case not exhaustive, in a general manner it can be mentioned that these ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin. The nature of these ingredients can be found in specialized books of perfumery, e.g. in S. Arctander (Perfume and Flavor Chemicals, Montclair N.J., USA 1969) or similar textbooks of reference, and a more detailed description thereof is not warranted here. A skilled person being able to select said ingredients on the basis of his general knowledge and according to the nature of the product to be perfumed and the desired olfactory effect.

The oil, especially when comprising a perfume, may possibly also contains a suitable solvent, in a quantity of up to 25% w/w of the oil, but preferably less than 10% w/w. The presence of a solvent may be useful to have a monophasic oil or to modulate surface tension of said oil. As example of suitable solvents, one may cite polar or non-polar low molecular weight solvent such as isoparaffins, paraffins, hydrocarbons, silicon oils, perfluorinated aliphatic ethers, glycol ethers, glycol ether esters, esters, or ketones. Non-restrictive examples of such solvents includes dimethicone or cyclomethicone, which are commercialized by Chemsil Silicon INC. under the trade names Cosmetic Fluid® 1288, and respectively Cosmetic Fluid® 1387, jojoba oil, perfluoroisobutyl methyl ether, diethyl phthalate, dipropylene glycol and isopropyl myristate.

Other possible ingredients of the oil are adjuvants or fixatives.

Concerning the physical properties of the oil, we have already mentioned that the latter must have a surface tension comprised in a specific range. According to a particular embodiment, the oil will have a surface tension comprised between 25 and 40 mN/m, at 25° C. Therefore, it is possible to use as oil a low polar perfume. By “low polar perfume” we mean here, for example, a perfume rich in highly hydrophobic perfuming ingredients or a perfume that contain only small amounts of polar solvents or completely free of polar solvents.

As low polar perfumes one can mention those containing from 5% w/w, or even 20% w/w, to 99% w/w of terpenes or/and from 5 to 30% w/w of musks; percentages being relative to the weight of the oil.

Said terpenes may be of wood or citrus origin and example of which are terpineol. or d-limonene. A non-restrictive example of musks is hexdecanolide.

As mentioned above, the oil represents between 3 to 30% of the microemulsion total weight. According to a particular embodiment the oil content represents preferably from 5 to 20% w/w, in respect to the microemulsion total weight.

The surfactant system, which is another mandator, element of the invention, may be described as consisting of a ionic fraction, representing more than 50% w/w of the surfactant system, and a non-ionic fraction, representing less than 50% w/w of the surfactant system.

The ionic fraction contains a single ionic surfactant or, according to an embodiment of the invention, a mixture of ionic surfactants. The ionic surfactants are of the anionic, cationic or amphoteric type.

Suitable anionic surfactants comprise the salts of C₆-C₂₄ mono- or di-sulfonic, alkylsulfuric, alkylarylsulfuric, alkylarylphosphate or carboxylic acids and also the polyethylene glycol co-polymers with sulfonic or carboxylic acids. Specific, but not limiting examples of said anionic surfactants are sodium, potassium, ammonium or mono-, di- or tri-ethanolammonium salts of C₆-C₁₂ dialkyl sulfosuccinic acids (such as sodium dioctyl-sulfosuccinate), C₇-C₂₄ alkarylsulfonic acids (such as sodium dodecyl benzenesulfonate), C₆-C₁₅ alkylsulfuric acid (such as sodium dodecylsulfate), C₁₀-C₂₀ acyl glutamic acid (such as sodium cocoyl glutamate), or polyethylene glycol/dimethicone sulfosuccinic acids (such as disodium PEG-12 dimethicone sulfosuccinate known under the trade name Mackanate® DC-50 from The McIntyre Group).

Suitable cationic surfactants comprise the salts of C₁₀-C₃₅ ammonium derivatives of fatty acids, alcohols, alkylamidoalkylmorpholine or amines and also the IPDI (isophorone diusocyanate) co-polymers with said ammonium derivatives or with fatty amines and possibly polyethylene glycols. Specific, but not-limiting examples, of said cationic surfactants are halides, sulfates or carboxylates of C₂₀₋₃₀ quaternary ammonium alkyl (such as hexadecyltrimethyl ammonium bromide or didodecylammonium bromide), C₁₋₄ alkyl N-cocoyl-L-arginate (such as DL-2-pyrrolidone-5-carboxylic acid salt of ethyl N-cocoyl-L-arginate commercialized by Ajinomoto Co., Inc. under the trade name Cae®), (C₁₀₋₂₀ amido) (C₁₋₄ alkyl) morpholine (such as isostearamidopropyl morpholine lactate), IPDI copolymers with N—C₁₀₋₂₀amido(C₁₋₄ alkyl)-N,N-di(C₁₋₄ alkyl)-N—(C₁₋₄ alkyl) Ammonium (such as bis(N-Ricinolemidopropyl-N,N-Dimethyl)/N-Ethyl Ammonium Sulfate/IPDI Copolymer also known under the trademark Polyquat® PPI-RC from ALZO) or polyethylene glycol/C₁₀-C₂₀ fatty alkyl amine/IPDI copolymers (such as the PEG Cocamine/IPDI Copolymeric surfactants also known under the trademark Polyderm® PPI-CA-15 from ALZO).

Suitable amphoteric surfactants comprise C₁₀-C₂₅ betaines, amphoacetates and imidazoline derivatives, as well as the polyethylene glycol/fatty amine glycine/IPDI copolymers. Specific, but not-limiting, examples of said amphoteric surfactants are the C₁₀-C₂₀ fatty amido C₂-C₅ alkyl betaines (such as cocoamidopropyl betaine), coco- and lauro-amphoacetates (such as sodium cocoamphoacetate known under the trade name Mackam® HPC-32 commercialized by McIntyre Group), and the polyethylene glycol/C₁₀-C₂₀ fatty alkyl amine/glycine/IPDI copolymers (such as PEG-13 soyamine-Glycine/IPDI Copolymer also known under the trademark Polytaine® PPI-SA-15 from ALZO).

The non-ionic fraction may contain a single non-ionic surfactant or, according to an embodiment of the invention, a mixture of non-ionic surfactants with an HLB value which comprises between 9 and 18. Suitable examples of said surfactants includes ethoxylated and/or propoxylated (C₅-C₁₂ alkyl)phenols ethers containing 5 to 20 EO or PO units (such as polyethylene glycol nonylphenyl ethers, polyethylene glycol octylphenyl ethers, also known under the generic tradename Polystep®), polyethylene glycol sorbitol ether containing 3 to 30 EO units (such as sorbitol esters with oleic, myristic, stearic, palmitic acid also known as those known under the tradenames Tweens® from ICI or Glycosperse® from LONZA), sucrose esters with C₈-C₂₀ fatty acid (such as sucrose esters with oleic, palmitic or stearic acid, such as Ryoto Sugar Ester M-1695 commercialized by Mitsubishi-Kagaku Foods Corporation), ethoxylated aliphatic C₆-C₂₀ alcohols containing 2 to 30 EO units (such as ethoxylated secondary C₆-C₂₀ alcohols), C₈-C₂₀ polyglyceryl esters (such as glycerol-polyethylene glycol oxystearate commercialized by BASF under the trade name Chromophor® CO40), polyethylene glycol and polypropylene glycol block copolymers (such as those known under the tradename Pluronics® from BASF), ethoxylated glycol ether containing 2 to 30 EO units (such as PEG-10 stearyl ether also known under the trade name Volpo® S-10 from CRODA), or polyethylene glycol mono- or -diester of aliphatic C₅-C₁₁ carboxylic acids containing 2 to 10 EO units (EO stands for ethylene oxide and PO stands for propylene oxide).

The amount of surfactant system needed to obtain a microemulsion according to the invention depends essentially on the amount of oil present and on the solubilizing-aid ingredient used. In a general manner, when the solubilizin-aid ingredient is used in amounts such as disclosed further below, we have noticed that the w/w oil/surfactant system ratio is comprised between 1 and 3. However, frequently it is possible to obtain a ratio comprised between 1.2, or even 1.5, and 3.

Another mandatory ingredient of the invention's microemulsions is the solubilizing-aid ingredient. By the expression “solubilizing-aid ingredient” we mean here an organic or inorganic salt, or a precursor thereof, of low molecular mass, e.g. below 400 g/mol. As solubilizing-aid ingredient it can also be used a mixture of said salts.

Said compounds, which per their nature are neither surfactants nor solvents, have been found to improve the solubility of organic compounds in water. In fact, and unexpectedly, these salts, or their precursors, are able to enhance the oil-solubilization capacity of the surfactant system. In other words, in the presence of a solubilizing-aid ingredient, as defined above, the same amount of surfactants is able to solubilize in the water phase more oil than if the solubilizing-aid ingredient was not present.

Moreover, the clarity of the microemulsion thus obtained is significantly and unexpectedly improved by the presence of the solubilizing-aid ingredient.

Thus, the presence of at least one of the above-mentioned salts has been found to be essential in order to ensure an oil/surfactant system ratio of at least 1, as well as a crystal-clear appearance, i.e. a high clarity or, if preferred, a low turbidity.

According to a particular embodiment of the invention, suitable salts are selected from the group consisting of sodium, potassium, magnesium and calcium salts of pyridine carboxylic acids, proline acid, pyrrolidone carboxylic acid, benzoic acid, L-lactic acid, L-ascorbic acid, bicarbonate, halide, succinic acid, oxalic acid, tartaric acid, citric acid, C₇ to C₁₁ derivatives of benzoic acids, hydroxyl-benzoic or amino-benzoic acids (such as the sodium salt of p-methyl-benzoic acid or of p-isopropyl-hydroxyl-benzoic acid), C₆ to C₁₆ derivatives of benzene- or naphthalene-sulfonic acids possibly hydroxylated (such as potassium toluene sulfonate or sodium butylnaphthalene sulfonate), C₁ to C₆ alkanoic acids (such as the sodium salt of pentanoic acid), and any mixture of said salts.

According to a particular embodiment of the invention, the solubilizing-aid ingredient may be advantageously chosen between the following compounds: pyrrolidone carboxylic acid sodium salt (also known as Ajidew NL-50 from Ajinomoto), sodium benzoate, sodium L-lactate, calcium L-ascorbate, sodium bicarbonate or di-sodium succinate. Any mixtures of said salts can also be used.

As mentioned above, the solubilizing-aid ingredient is present in an amount comprised between 0.1 and 10% w/w in respect to the total weight of the microemulsion. According to a particular embodiment, said amount is advantageously comprised between 0.1 and 5% w/w, or even between 0.1 and 2% w/w.

Concerning the fourth components of the present microemulsion, i.e. water, it is useful to mention that it is preferable to use de-ionized water.

The invention's microemulsion can also comprise, as optional components, one or more ingredients such as colorants, anti-microbial agents, antioxidants, preservatives, chelating agents or UV-inhibitors. Such types of materials are well known to a person skilled in the art and do not need a more detailed description. Whenever said ingredients are added to the microemulsion, then they will represent no more than 3% w/w, or even 2% w/w, the percentages being relative to the total weight of the microemulsion.

The invention's microemulsion can be prepared according to any method known in the art. A suitable method consists in dissolving into the water the surfactant system, to form a clear micellar solution. To the resulting micellar solution are added under gentle stirring the solubilizing-aid ingredient, and whenever necessary the optional ingredients to form an initial pre-microemulsion. Under gentle mixing the resulting pre-microemulsion can easily solubilize the corresponding amount of perfume to form an isotropic clear, single-phase microemulsion product. High mechanical forces such as shear forces are not necessary to manufacture the present microemulsion.

As anticipated above, the microemulsion of the invention is particularly suitable for the manufacture of consumer articles capable of dispensing a perfume in the surrounding space. Said consumer articles are also an object of the present invention.

A suitable consumer article comprises a microemulsion as described above together with a suitable container and possibly a means to produce an aerosol. Non-limiting examples of such consumer article are room deodorants, or air fresheners, as well as hair or skin preparations, such as fine perfumery articles.

In another embodiment, said consumer articles comprise a microemulsion according to the invention and a consumer product base. For the sake of clarity, it has to be mentioned that, by “consumer product base” we mean here a consumer product, i.e. a consumable product such as a cosmetic preparation, or a part of said consumer product. Therefore, a consumer article according to the present embodiment of the invention comprises at least a part of the whole formulation corresponding to a desired consumer product and an olfactive effective amount of the invention's microemulsion. A suitable consumer product base is, for examples, a surface cleaning product, an hygiene product. an hair care product such as shampoos, a body-care product, a cosmetic preparation, a fabric refreshers, an ironing water or a wipe.

The nature and type of the constituents of the consumer product do not warrant a more detailed description here, which in any case would not be exhaustive, the skilled person being able to select them on the basis of its general knowledge and according to the nature and the desired effect of said product.

A further object of the present invention is the use of a microemulsion according to the invention as a perfuming ingredient. In other, a method to confer, enhance, improve or modify the odor properties of a composition or of an article, which method comprise adding to said composition or article an olfactive effective amount of a microemulsion as defined above.

### EXAMPLES

The invention will now be described in further detail by way of the following examples.

### Example 1

### Preparation of Perfume-in Water Microemulsion According to the Invention

A perfume containing almost 70% of terpenes was obtained by ad-mixing the following ingredients:

| Ingredient | Parts by weight |
|---|---|
| Benzyl salicylate | 21.00 |
| Exaltolide ®¹⁾ | 15.00 |
| Grapefruit oil | 200.00 |
| Iso E Super²⁾ | 21.00 |
| Lilial ®³⁾ | 20.00 |
| Mandarin oil sfuma | 55.00 |
| (Z)-3-Methyl-2-(2-pentenyl)-2-cyclopenten-1-one | 1.00 |
| Hedione ®⁴⁾ | 100.00 |
| Nerol | 10.00 |
| Orange oil | 150.00 |
| Spearmint oil | 2.00 |
| Styrallyl acetate | 5.00 |
| Total | 600.00 |

¹⁾Pentadecanolide; origin: Firmenich SA, Geneva, Switzerland
²⁾1-(octahydro-2,3,8,8-tetramethyl-2-naphthalenyl)-1-ethanone origin: International Flavors & Fragrances, USA
³⁾3-(4-tert-butylphenyl)-2-methylpropanal; origin: Givaudan-Roure SA, Vernier, Switzerland
⁴⁾Methyl dihydrojasmonate; origin: Firmenich SA, Geneva, Switzerland

In a general way, a microemulsion according to the invention was obtained, in a first step, by mixing together in a beaker the de-ionized water, the surfactants, and the solubilizing-aid ingredient. The mixture is gently stirred at room temperature for a few minutes by means of any common mixing device, such as a magnetic stirring. Possibly, during the stirring the mixture may be protected by a nitrogen blanket. In the second step, the fragrance is added under stirring over the above water solution. The microemulsion was formed in few minutes. Usually a clear product was obtained in less than 10 minutes, sometimes even instantly.

Following this general procedure, it was obtained a microemulsion according to Table 1 and 2, and having a oil/surfactant system ratio of 1.71:

TABLE 1
formulation of the microemulsion

| Ingredient | Parts by weight |
|---|---|
| Perfume | 15.15 |
| Surfactant system: | 8.87 |
| PEO (20) Sorbitan monooleate | 2.07 |
| PEO (20) Sorbitan monolaurate | 0.65 |
| Solubilisant LRI¹⁾ | 1.63 |
| Sodium Dioctyl Sulfosuccinate | 3.87 |
| Sodium Dodecylsulfate | 0.65 |
| Solubilizing-aid ingredient: | 1.13 |
| Sodium Benzoate | 0.40 |
| Ajidew NL-50²⁾ | 0.40 |
| Sodium L-lactate | 0.33 |
| Optional ingredient: | 0.35 |
| Glydant Plus³⁾ | 0.35 |
| De-ionized water | 74.50 |
| Total | 100.00 |

¹⁾89.7% Aqueous Blend of non-ionic surfactants from LCW
²⁾Sodium Pyrrolidone Carboxylic Acid 50% aqueous solution from Ajinimoto Inc.
³⁾87% Aqueous solution, origin: Lonza

TABLE 2
physical properties of the microemulsion described in Table 1

| | |
|---|---|
| Clarity¹⁾ | |
| T = 25° C. | 75.1 NTU (oil droplet size: 24.5 nm) |
| T = 0-2° C. | 76.9 NTU (oil droplet size: 26.7 nm) |
| Surface tension (mN/m) | 26.44 ± 0.05 |
| Temperature Stability | from 0° C. to 60° C. |
| Viscosity (25° C.)²⁾ | 7.02 cPs |

¹⁾NTU is Nephelometric Turbidity Unit, measured on a Turbidimeter VWR Model 66120-200, with a tungsten lamp and 2 photo voltaic cells centered at 90° to the incident light, response between 400 and 600 nm.
²⁾Measured with a Brookfield Viscometer with spindle YULA-15 at 30 rpm

### Example 2

### Preparation of Perfume-in Water Microemulsion According to the Invention

Following the same procedure as described in Example 1, it was obtained a microemulsion according to Table 3 and 4, and having a oil/surfactant system ratio of 1.09:

TABLE 3
formulation of the microemulsion

| Ingredient | Parts by weight |
|---|---|
| Perfume¹⁾ | 10.16 |
| Surfactant system: | 9.34 |
| PEO (20) Sorbitan monooleate | 2.17 |
| PEO (20) Sorbitan monolaurate | 0.68 |
| Solubilisant LRI¹⁾ | 1.72 |
| Sodium Dioctyl Sulfosuccinate | 4.09 |
| Sodium Dodecylsulfate | 0.68 |
| Solubilizing-aid ingredient: | 1.36 |
| Ajidew NL-50¹⁾ | 1.36 |
| De-ionized water | 79.14 |
| Total | 100.00 |

¹⁾as in Example 1.

TABLE 4
physical properties of the microemulsion described in table 3

| | |
|---|---|
| Clarity¹⁾ | |
| T = 25° C. | 27.6 NTU (oil droplet size: 14.5 nm) |
| T = 0-2° C. | 30.3 NTU (oil droplet size: 15.9 nm) |
| Surface tension (mN/m) | 26.58 ± 0.04 |
| Temperature Stability | from 0° C. to 65° C. |
| Viscosity (25° C.)²⁾ | 4.14 cPs |

¹⁾as in Example 1.

### Example 3

### Preparation of Perfume-in Water Microemulsion According to the Invention

A perfume was obtained by admixing the following ingredients:

| Ingredients | Parts by weight |
|---|---|
| Citronellyl acetate | 3 |
| Geranyl acetate | 9 |
| Linalyl acetate | 276 |
| 10% *Aldehyde C10 | 3 |
| 10% *Aldehyde C12 | 12 |
| Methyl anthranilate | 16 |
| Bergamote essential oil | 226 |
| Cetalox ®¹⁾ | 5 |
| Lemon essential oil | 318 |
| Dihydromyrcenol²⁾ | 60 |
| Dipropylene glycol | 20 |
| 10% *Elemi³⁾ | 20 |
| Fleuria 41063 B⁴⁾ | 3 |
| Ethyl linalol | 66 |
| 10% *3-(4-Methoxyphenyl)-2-methylpropanal⁴⁾ | 30 |
| Geraniol | 6 |
| 50% *Habanolide ®⁵⁾ | 130 |
| Hedione ®⁶⁾ | 215 |
| Hedione ® HC⁷⁾ | 72 |
| 10% **Indol | 12 |
| Iso E super⁸⁾ | 85 |
| Lavandin grosso essential oil | 26 |
| 1% *Liffarome ®⁹⁾ | 20 |
| Linalol | 40 |
| Mandarine sfuma essential oil | 5 |
| 10% *Spearmint essential oil | 30 |
| Neroli bigarade essential oil | 130 |
| Orange essential oil | 80 |
| Phenethylol | 9 |
| Petitgrain essential oil | 63 |
| Pipol | 5 |
| Rosemary essential oil | 16 |
| Terpineol | 9 |
| Violet essential oil | 50 |
| 1% *Zestover¹⁰⁾ | 30 |
| Total | 2100 |

*in dipropylene glycol (DIPG)
**in triethanolamine
¹⁾8,12-epoxy-13,14,15,16-tetranorlabdane
²⁾origin: International Flavors and Fragrances, USA
³⁾5-Allyl-1,2,3-trimethoxybenzene; origin: Calchauvet, Grasse, France
⁴⁾origin: Firmenich SA, Geneva, Switzerland
⁵⁾pentadecenolide; origin: Firmenich SA, Geneva, Switzerland
⁶⁾Methyl dihydrojasmonate; origin: Firmenich SA, Geneva, Switzerland
⁷⁾Methyl dihydrojasmonate with a high content of cis isomer; origin: Firmenich SA, Geneva, Switzerland
⁸⁾1-(octahydro-2,3,8,8-tetramethyl-2-naphthalenyl)-1-ethanone; origin: International Flavors and Fragrances, USA
⁹⁾3-Hexenyl methyl carbonate; origin: International Flavors and Fragrances, USA
¹⁰⁾9-decen-1-ol; origin: International Flavors and Fragrances, USA

Following the same procedure as described in Example 1, and the perfume above described, it was obtained a microemulsion according to Table 5 and 6, and having a oil/surfactant system ratio of 1.71:

TABLE 5
formulation of the microemulsion

| Ingredient | Parts by weight |
|---|---|
| Perfume | 15.18 |
| Surfactant system: | 8.89 |
| PEO (20) Sorbitan monooleate | 2.08 |
| PEO (20) Sorbitan monolaurate | 0.64 |
| Solubilisant LRI¹⁾ | 1.64 |
| Sodium Dioctyl Sulfosuccinate | 3.89 |
| Sodium Dodecylsulfate | 0.64 |
| Solubilizing-aid ingredient: | 0.98 |
| Sodium Benzoate | 0.40 |
| Ajidew NL-50¹⁾ | 0.33 |
| Sodium L-lactate | 0.25 |
| Optional ingredient: | 0.37 |
| Glydant Plus³⁾ | 0.37 |
| De-ionized water | 74.58 |
| Total | 100.00 |

¹⁾as in Example 1.

TABLE 6
physical properties of the microemulsion described in table 5

| | |
|---|---|
| Clarity¹⁾ | |
| T = 25° C. | 48.4 NTU (oil droplet size: 28.3 nm) |
| T = 0-2° C. | 49.5 NTU (oil droplet size: 29.8 nm) |
| Surface tension (mN/m) | 26.44 ± 0.05 |
| Temperature Stability | from 0° C. to 57° C. |
| Viscosity (25° C.)²⁾ | 7.98 cPs |

¹⁾as in Example 1.

### Example 4

### Preparation of Perfume-in Water Microemulsion According to the Invention

Following the same procedure as described in Example 1, it was obtained a microemulsion according to Table 7 and 8, and having a oil/surfactant system ratio of 1.09:

TABLE 7
formulation of the microemulsion

| Ingredient | Parts by weight |
|---|---|
| Perfume²⁾ | 10.31 |
| Surfactant system: | 9.46 |
| PEO (20) Sorbitan monooleate | 2.20 |
| PEO (20) Sorbitan monolaurate | 0.69 |
| Solubilisant LRI¹⁾ | 1.75 |
| Sodium Dioctyl Sulfosuccinate | 4.13 |
| Sodium Dodecylsulfate | 0.69 |
| Solubilizing-aid ingredient: | 0.67 |
| Sodium Benzoate | 0.40 |
| Ajidew NL-50¹⁾ | 0.27 |
| Optional ingredient: | 0.34 |
| Glydant Plus¹⁾ | 0.34 |
| De-ionized water | 79.22 |
| Total | 100.00 |

¹⁾as in Example 1
²⁾as in example 3

TABLE 8
physical properties of the microemulsion described in table 3

| | |
|---|---|
| Clarity¹⁾ | |
| T = 25° C. | 14.0 NTU (oil droplet size: 15.9 nm) |
| T = 0-2° C. | 14.2 NTU (oil droplet size: 18.0 nm) |
| Surface tension (mN/m) | 26.66 ± 0.05 |
| Temperature Stability | from 0° C. to 70° C. |
| Viscosity (25° C.)²⁾ | 4.50 cPs |

¹⁾as in Example 1

What is claimed is:

1. A VOC-free o/w microemulsion comprising:
A) from 3 to 30% w/w of an oil having a surface tension, at 25° C., of at least 25 mN/m;
B) a surfactant system, comprising one ore more ionic surfactants, in a quantity of at least 50% w/w of the surfactant system, and one or more non-ionic surfactant, in a quantity of at most 50% w/w of the surfactant system; the w/w oil/surfactant system ratio being comprised between 1 and 3;
C) from 0.1 to 10% w/w of a solubilizing-aid ingredient selected from the group consisting of the ammonium, alkaline and alkaline earth salts of C₁ to C₁₅ mono- and di-carboxylic acids derivatives, bicarbonates, halogenates, thiocyanates, and mixtures of said salts; and
D) at least 60% of water;
said microemulsion having a clarity comprised between 0 and 90 NTU, when measured between 400 and 600 nm in a 2.5 cm cell at 25° C.

2. A microemulsion according to claim 1, wherein the VOC is selected from the group consisting of C₁-C₅ alkanols and C₁-C₅ alkanediols.

3. A microemulsion according to claim 1, wherein the clarity is comprised between 0 and 50 NTU, when measured between 400 and 600 nm in a 2.5 cm cell at 25° C.

4. A microemulsion according to claim 1, wherein said oil represents from 5 to 20% w/w of the microemulsion.

5. A microemulsion according to claim 1, wherein said oil comprises at least 75% of a perfume.

6. A microemulsion according to claim 5, wherein said oil comprises at least 90% of a perfume.

7. A microemulsion according to claim 5, wherein said perfume contains from 5% w/w to 99% w/w of terpenes or from 5 to 30% w/w of musks.

8. A microemulsion according to claim 1, wherein the w/w oil/surfactant system ratio is comprised betaeen 1.2 and 3.

9. A microemulsion according to claim 1, wherein
a) the anionic surfactants are selected from the group consisting of sodium, potassium, ammonium and mono-, di- and tri-ethanolammonium salts of C₆-C₁₂ dialkyl sulfosuccinic acids, C₇-C₂₄ alkarylsulfonic acids, C₆-C₁₅ alkylsulfuric acid, C₁₀-C₂₀ acyl glutamic acid, and polyethylene glycol/dimethicone sulfosuccinic acids;
b) the cationic surfactants are selected from the group consisting of halides, sulfates or carboxylates of C₂₀₋₃₀ quaternary ammonium alkyl, C₁₋₄ alkyl N-cocoyl-L-arginate, (C₁₀₋₂₀ amido) (C₁₋₄ alkyl) morpholine, IPDI copolymers with N—C₁₀₋₂₀amido(C₁₋₄ alkyl)-N,N-di(C₁₋₄ alkyl)-N—(C₁₋₄ alkyl) Ammonium, and polyethylene glycol/C₁₀-C₂₀ fatty alkyl amine/IPDI copolymers;
c) the amphoteric surfactants are selected from the group consisting of C₁₀-C₂₀ fatty amido C₂-C₅ alkyl betaines, coco- and lauro-amphoacetates and polyethylene glycol/C₁₀-C₂₀ fatty alkyl amine/glycine/IPDI copolymers;
d) the non-ionic surfactants are selected from the group consisting of ethoxylated and propoxylated (C₅-C₁₂ alkyl)phenols ethers containing 5 to 20 EO or PO units, polyethylene glycol sorbitol ether containing 3 to 30 EO units, sucrose esters with C₈-C₂₀ fatty acid, ethoxylated aliphatic C₆-C₂₀ alcohols containing 2 to 30 EO units, C₈-C₂₀ polyglyceryl esters, polyethylene glycol and polypropylene glycol block copolymers, ethoxylated glycol ether containing 2 to 30 EO units, and polyethylene glycol mono- and -diester of aliphatic C₅-C₁₁ carboxylic acids containing 2 to 10 EO units.

10. A microemulsion according to claim 1, wherein the solubilizing-aid ingredient is selected from the group consisting of sodium, potassium, magnesium and calcium salts of pyridine carboxylic acids, proline acid, pyrrolidone carboxylic acid, benzoic acid, L-lactic acid, L-ascorbic acid, bicarbonate, halide, succinic acid, oxalic acid, tartaric acid, citric acid, C₇ to C₁₁ derivatives of benzoic acids, hydroxyl-benzoic or amino-benzoic acids, C₆ to C₁₆ derivatives of benzene- or naphthalene-sulfonic acids possibly hydroxylated, C₁ to C₆ alkanoic acids, and any mixture of said salts

11. A microemulsion according to claim 1, wherein the solubilizing-aid ingredient is selected from the group consisting of pyrrolidone carboxylic acid sodium salt, sodium benzoate, sodium L-lactate, calcium L-ascorbate, sodium bicarbonate, di-sodium succinate and any mixture of said salts.

12. A microemulsion according to claim 1, wherein the solubilizing-aid ingredient represents from 0.1 to 3% w/w of the microemulsion

13. A microemulsion according to claim 1, wherein said microemulsion is stable for at least 30 days, at temperatures between 2° C. and 60° C.

14. A microemulsion according to claim 1, wherein said microemulsion has a surface tension, at 25° C., of at most 30 mN/m.

15. A consumer article comprising a microemulsion, as defined in claim 1, together with a container and possibly means to produce an aerosol.

16. A consumer article according to claim 15, in the form of a room deodorant or a hair or skin preparation.

17. A consumer article comprising a microemulsion, as defined in claim 1, and a consumer article base.

18. A method to confer, enhance, improve or modify the odor properties of a composition or of an article, which method comprise adding to said composition or article an olfactive effective amount of a microemulsion as defined in claim 1.
